(19) United States
(12) Patent Application Publication
(10) Pub. No.: US 2008/0113051 A1
(43) Pub. Date: May 15, 2008

(54) METHODS FOR ALLEVIATING TATTOO PAIN

(75) Inventor: Terrence J. Hunt, Corona, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 11/559,052

(22) Filed: Nov. 13, 2006

(51) Int. Cl. A61K 35/74; A61P 25/00

(52) U.S. Cl. 424/780

(57) ABSTRACT

The present invention features a method for alleviating pain associated with a placement or removal of a tattoo. In some embodiments, the method comprises the step of locally administering a neurotoxin (e.g., a botulinum toxin) to the anatomical site at where the tattoo is to be placed or removed.

The present invention relates to methods for alleviating a pain associated with tattoo placement and/or tattoo removal.

Tattooing has been around since ancient times and its presence in mainstream society is growing. It has been estimated that about 25 million people in the United States have at least one tattoo. With the current popularity of “body art”, over 250,000 people are being tattooed each year. The average age of procuring a tattoo is 18 years, often as a statement of love, anger, sexuality, individuality, and/or group identity.

Permanent tattoos are applied by injecting color pigments into the dermal layer of the skin using a solid, round-tip needle attached to a motorized instrument that holds up to 14 needles attached to the pigments. The pigments are injected into the dermis layer of the skin at a rate of 15 to 3,000 times per minute.

Most tattoo artists use an electrically powered, vertical, vibrating instrument to inject the tattoo pigment. The instrument injects pigment at 50 to 30,000 times per minute into the dermis layer of skin, at a depth of 1/64 to 1/16 of an inch. A single needle outlines the tattoo and the design is then filled in with five to seven needles in a needle bar.

As the popularity, visibility, and social acceptability of tattooing increases in the United States and elsewhere, so does the demand for fast, safe, and effective tattoo removal. The goal of tattoo removal is to completely clear, or at least dramatically lighten, tattoos without adversely altering the skin's appearance and texture. There are several tattoo removal techniques.

Tattoo Removal

The method of choice will depend upon the size of the tattoo and its location as well as the length of time it has been on the skin.

Laser

Recently, laser surgery has become one of the best methods of tattoo removal. Today, the Q-SWITCHED ND: YAG®, Q-SWITCHED ALEXANDRITE® and the Q-SWITCHED RUBY® are among the most frequently used lasers today for the removal of unwanted tattoos. They are all employed in a similar manner. If necessary, a cream to numb the skin can be applied prior to the treatment. Pulses of light from the laser are directed onto the tattoo breaking up the tattoo pigment. Over the next several weeks the body's scavenger cells remove the treated pigmented areas. More than one treatment is usually necessary to remove all of the tattoo. Large areas e.g., “full sleeves” wherein the entire arm is covered or full back tattoos require multiple sessions requiring the recipient to endure many hours of often excruciating pain.

Dermabrasion

Another popular method of tattoo removal is called dermabrasion in which a small portion of the tattoo is sprayed with a solution that freezes the area. The tattoo is then “sanded” with a rotary abrasive instrument causing the skin to peel. Because some bleeding is likely to occur, a dressing is immediately applied to the area.

Salabrasion

Salabrasion is a method still sometimes used today to remove tattoos. As with the other methods, a local anesthetic is used on and around the tattooed area after which a solution of ordinary tap water dipped in table salt is applied. An abrading apparatus such as the one used with dermabrasion, or an even simpler device such as a wooden block wrapped in gauze, is used to vigorously abrade the area. When the area becomes deep red in color, a dressing is applied.

Excision

Excision may be the technique used to remove small tattoos. The advantage of this method is that the entire tattoo can be removed. With larger tattoos, however, it may be necessary to excise in stages, removing the center of it initially and the sides at a later date.

Excision involves an injection of a local anesthetic to numb the area after which the tattoo is removed surgically. The edges are then brought together and sutured. With this procedure, there is minimal bleeding which is easily controlled with electrocautery. In some cases involving large tattoos, a skin graft taken from another part of the body may be necessary.

Pain

The placement or removal process of a tattoo may cause excruciating pain. The genitals are most sensitive to pain associated with a tattoo placement or removal. The sternum, ribs, hands, and feet are also sensitive. The ankles, lower back, neck, under arm, around the groin area, and head are not as sensitive to pain. The least sensitive areas are the upper arms or forearms, calves, shoulder blade, outer thigh, and bottom.

Various pain alleviating techniques are currently being used in conjunction with the tattoo placement or removal process. For example, the administration of lidocaine is often used in conjunction with a placement or removal of a tattoo. However, the current pain alleviating techniques are not satisfactory. For example, lidocaine treatment could provide pain relief for about several hours, while a pain associated with a tattoo placement or removal can lasts several weeks. Thus, there is a need for better techniques for alleviating a pain associated with a tattoo placement or removal.

Botulinum Toxin

The genus Clostridium has more than one hundred and twenty seven species, grouped according to their morphology and functions. The anaerobic, gram positive bacterium Clostridium botulinum produces a potent polypeptide Clostridial toxin, botulinum toxin, which causes a neuroparalytic illness in humans and animals referred to as botulism. The spores of Clostridium botulinum are found in soil and can grow in improperly sterilized and sealed food containers of home based canneries, which are the cause of many of the cases of botulism. The effects of botulism typically appear 18 to 36 hours after eating the foodstuffs infected with a Clostridium botulinum culture or spores. The botulinum toxin can apparently pass unattenuated through the lining of the gut and attack peripheral motor neurons. Symptoms of botulinum toxin intoxication can progress from difficulty walking, swallowing, and speaking to paralysis of the respiratory muscles and death.

Botulinum toxin type A is the most lethal natural biological agent known to man. About 50 picograms of a commercially available botulinum toxin type A (purified Clostridial toxin complex)¹ is a LD₅₀ in mice (i.e. 1 unit). One unit of BOTOX® contains about 50 picograms (about 56 attomoles) of botulinum toxin type A complex. Interestingly, on a molar basis, botulinum toxin type A is about 1.8 billion times more lethal than diphtheria, about 600 million times more lethal than sodium cyanide, about 30 million times more lethal than cobra toxin and about 12 million times more lethal than cholera. Singh, Critical Aspects of Bacterial Protein Toxins, pages 63-84 (chapter 4) of Natural Toxins II, edited by B. R. Singh et al., Plenum Press, New York (1996) (where the stated LD₅₀ of botulinum toxin type A of 0.3 ng equals 1 U is corrected for the fact that about 0.05 ng of BOTOX® equals 1 unit). One unit (U) of botulinum toxin is defined as the LD₅₀ upon intraperitoneal injection into female Swiss Webster mice weighing 18 to 20 grams each. ¹Available from Allergan, Inc., of Irvine, Calif. under the tradename BOTOX® in 100 unit vials

Seven immunologically distinct botulinum Clostridial toxins have been characterized, these being respectively botulinum Clostridial toxin serotypes A, B, C₁, D, E, F and G each of which is distinguished by neutralization with type-specific antibodies. The different serotypes of botulinum toxin vary in the animal species that they affect and in the severity and duration of the paralysis they evoke. For example, it has been determined that botulinum toxin type A is 500 times more potent, as measured by the rate of paralysis produced in the rat, than is botulinum toxin type B. Additionally, botulinum toxin type B has been determined to be non-toxic in primates at a dose of 480 U/kg which is about 12 times the primate LD₅₀ for botulinum toxin type A. Moyer E et al., Botulinum Toxin Type B: Experimental and Clinical Experience, being chapter 6, pages 71-85 of “Therapy With Botulinum Toxin”, edited by Jankovic, J. et al. (1994), Marcel Dekker, Inc. Botulinum toxin apparently binds with high affinity to cholinergic motor neurons, is translocated into the neuron and blocks the release of acetylcholine.

Regardless of serotype, the molecular mechanism of toxin intoxication appears to be similar and to involve at least three steps or stages. In the first step of the process, the toxin binds to the presynaptic membrane of the target neuron through a specific interaction between the heavy chain, H chain, and a cell surface receptor; the receptor is thought to be different for each type of botulinum toxin and for tetanus toxin. The carboxyl end segment of the H chain, H_{C}, appears to be important for targeting of the toxin to the cell surface.

In the second step, the toxin crosses the plasma membrane of the poisoned cell. The toxin is first engulfed by the cell through receptor-mediated endocytosis, and an endosome containing the toxin is formed. The toxin then escapes the endosome into the cytoplasm of the cell. This step is thought to be mediated by the amino end segment of the H chain, H_{N}, which triggers a conformational change of the toxin in response to a pH of about 5.5 or lower. Endosomes are known to possess a proton pump which decreases intra-endosomal pH. The conformational shift exposes hydrophobic residues in the toxin, which permits the toxin to embed itself in the endosomal membrane. The toxin (or at a minimum the light chain) then translocates through the endosomal membrane into the cytoplasm.

The last step of the mechanism of botulinum toxin activity appears to involve reduction of the disulfide bond joining the heavy chain, H chain, and the light chain, L chain. The entire toxic activity of botulinum and tetanus toxins is contained in the L chain of the holotoxin; the L chain is a zinc (Zn++) endopeptidase which selectively cleaves proteins essential for recognition and docking of neurotransmitter-containing vesicles with the cytoplasmic surface of the plasma membrane, and fusion of the vesicles with the plasma membrane. Tetanus Clostridial toxin, botulinum toxin types B, D, F, and G cause degradation of synaptobrevin (also called vesicle-associated membrane protein (VAMP)), a synaptosomal membrane protein. Most of the VAMP present at the cytoplasmic surface of the synaptic vesicle is removed as a result of any one of these cleavage events. Botulinum toxin serotype A and E cleave SNAP-25. Botulinum toxin serotype C₁ was originally thought to cleave syntaxin, but was found to cleave syntaxin and SNAP-25. Each of the botulinum toxins specifically cleaves a different bond, except botulinum toxin type B (and tetanus toxin) which cleave the same bond.

Although all the botulinum toxins serotypes apparently inhibit release of the neurotransmitter acetylcholine at the neuromuscular junction, they do so by affecting different neurosecretory proteins and/or cleaving these proteins at different sites. For example, botulinum types A and E both cleave the 25 kiloDalton (kD) synaptosomal associated protein (SNAP-25), but they target different amino acid sequences within this protein. Botulinum toxin types B, D, F and G act on vesicle-associated protein (VAMP, also called synaptobrevin), with each serotype cleaving the protein at a different site. Finally, botulinum toxin type C₁ has been shown to cleave both syntaxin and SNAP-25. These differences in mechanism of action may affect the relative potency and/or duration of action of the various botulinum toxin serotypes. Apparently, a substrate for a botulinum toxin can be found in a variety of different cell types. See e.g. Gonelle-Gispert, C., et al., SNAP-25a and −25b isoforms are both expressed in insulin-secreting cells and can function in insulin secretion, Biochem J. 1;339 (pt 1):159-65:1999, and Boyd R. S. et al., The effect of botulinum Clostridial toxin-B on insulin release from a ∃-cell line, and Boyd R. S. et al., The insulin secreting ∃-cell line, HIT-15, contains SNAP-25 which is a target for botulinum Clostridial toxin-A, both published at Mov Disord, 10(3):376:1995 (pancreatic islet B cells contains at least SNAP-25 and synaptobrevin).

The molecular weight of the botulinum toxin protein molecule, for all seven of the known botulinum toxin serotypes, is about 150 kD. Interestingly, the botulinum toxins are released by Clostridial bacterium as complexes comprising the 150 kD botulinum toxin protein molecule along with associated non-toxin proteins. Thus, the botulinum toxin type A complex can be produced by Clostridial bacterium as 900 kD, 500 kD and 300 kD forms. Botulinum toxin types B and C₁ is apparently produced as only a 700 kD or 500 kD complex. Botulinum toxin type D is produced as both 300 kD and 500 kD complexes. Finally, botulinum toxin types E and F are produced as only approximately 300 kD complexes. The complexes (i.e. molecular weight greater than about 150 kD) are believed to contain a non-toxin hemaglutinin protein and a non-toxin and non-toxic nonhemaglutinin protein. These two non-toxin proteins (which along with the botulinum toxin molecule comprise the relevant Clostridial toxin complex) may act to provide stability against denaturation to the botulinum toxin molecule and protection against digestive acids when toxin is ingested. Additionally, it is possible that the larger (greater than about 150 kD molecular weight) botulinum toxin complexes may result in a slower rate of diffusion of the botulinum toxin away from a site of intramuscular injection of a botulinum toxin complex.

All the botulinum toxin serotypes are made by Clostridium botulinum bacteria as inactive single chain proteins which must be cleaved or nicked by proteases to become neuroactive. The bacterial strains that make botulinum toxin serotypes A and G possess endogenous proteases and serotypes A and G can therefore be recovered from bacterial cultures in predominantly their active form. In contrast, botulinum toxin serotypes C₁, D, and E are synthesized by nonproteolytic strains and are therefore typically unactivated when recovered from culture. Serotypes B and F are produced by both proteolytic and nonproteolytic strains and therefore can be recovered in either the active or inactive form. However, even the proteolytic strains that produce, for example, the botulinum toxin type B serotype only cleave a portion of the toxin produced. The exact proportion of nicked to unnicked molecules depends on the length of incubation and the temperature of the culture. Therefore, a certain percentage of any preparation of, for example, the botulinum toxin type B toxin is likely to be inactive, possibly accounting for a lower potency of botulinum toxin type B as compared to botulinum toxin type A. The presence of inactive botulinum toxin molecules in a clinical preparation will contribute to the overall protein load of the preparation, which has been linked to increased antigenicity, without contributing to its clinical efficacy.

Botulinum toxins and toxin complexes can be obtained from, for example, List Biological Laboratories, Inc., Campbell, Calif.; the Centre for Applied Microbiology and Research, Porton Down, U.K.; Wako (Osaka, Japan), as well as from Sigma Chemicals of St Louis, Mo. Commercially available botulinum toxin containing pharmaceutical compositions include BOTOX® (Botulinum toxin type A Clostridial toxin complex with human serum albumin and sodium chloride) available from Allergan, Inc., of Irvine, Calif. in 100 unit vials as a lyophilized powder to be reconstituted with 0.9% sodium chloride before use), Dysport® (Clostridium botulinum type A toxin haemagglutinin complex with human serum albumin and lactose in the formulation), available from Ipsen Limited, Berkshire, U.K. as a powder to be reconstituted with 0.9% sodium chloride before use), and MyoBloc™ (an injectable solution comprising botulinum toxin type B, human serum albumin, sodium succinate, and sodium chloride at about pH 5.6, available from Elan Corporation, Dublin, Ireland).

The success of botulinum toxin type A to treat a variety of clinical conditions has led to interest in other botulinum toxin serotypes. Additionally, pure botulinum toxin has been used to treat humans. See e.g. Kohl A., et al., Comparison of the effect of botulinum toxin A (BOTOX(R)) with the highly-purified Clostridial toxin (NT 201) in the extensor digitorum brevis muscle test, Mov Disord 2000; 15(Suppl 3):165. Hence, a pharmaceutical composition can be prepared using a pure botulinum toxin.

The type A botulinum toxin is known to be soluble in dilute aqueous solutions at pH 4-6.8. At pH above about 7 the stabilizing nontoxic proteins dissociate from the Clostridial toxin, resulting in a gradual loss of toxicity, particularly as the pH and temperature rise. Schantz E. J., et al Preparation and characterization of botulinum toxin type A for human treatment (in particular pages 44-45), being chapter 3 of Jankovic, J., et al, Therapy with Botulinum Toxin, Marcel Dekker, Inc (1994).

The botulinum toxin molecule (about 150 kDa), as well as the botulinum toxin complexes (about 300-900 kDa), such as the toxin type A complex are also extremely susceptible to denaturation due to surface denaturation, heat, and alkaline conditions. Inactivated toxin forms toxoid proteins which may be immunogenic. The resulting antibodies can render a patient refractory to toxin injection.

In vitro studies have indicated that botulinum toxin inhibits potassium cation induced release of both acetylcholine and norepinephrine from primary cell cultures of brainstem tissue. Additionally, it has been reported that botulinum toxin inhibits the evoked release of both glycine and glutamate in primary cultures of spinal cord neurons and that in brain synaptosome preparations botulinum toxin inhibits the release of each of the neurotransmitters acetylcholine, dopamine, norepinephrine (Habermann E., et al., Tetanus Toxin and Botulinum A and C Clostridial toxins Inhibit Noradrenaline Release From Cultured Mouse Brain, J Neurochem 51(2);522-527:1988) CGRP, substance P and glutamate (Sanchez-Prieto, J., et al., Botulinum Toxin A Blocks Glutamate Exocytosis From Guinea Pig Cerebral Cortical Synaptosomes, Eur J. Biochem 165;675-681:1987. Thus, when adequate concentrations are used, stimulus-evoked release of most neurotransmitters is blocked by botulinum toxin. See e.g. Pearce, L. B., Pharmacologic Characterization of Botulinum Toxin For Basic Science and Medicine, Toxicon 35(9);1373-1412 at 1393; Bigalke H., et al., Botulinum A Clostridial toxin Inhibits Non-Cholinergic Synaptic Transmission in Mouse Spinal Cord Neurons in Culture, Brain Research 360;318-324:1985; Habermann E., Inhibition by Tetanus and Botulinum A Toxin of the release of [³H]Noradrenaline and [³H]GABA From Rat Brain Homogenate, Experientia 44;224-226:1988, Bigalke H., et al., Tetanus Toxin and Botulinum A Toxin Inhibit Release and Uptake of Various Transmitters, as Studied with Particulate Preparations From Rat Brain and Spinal Cord, Naunyn-Schmiedeberg's Arch Pharmacol 316;244-251:1981, and; Jankovic J. et al., Therapy With Botulinum Toxin, Marcel Dekker, Inc., (1994), page 5.

Botulinum toxin type A can be obtained by establishing and growing cultures of Clostridium botulinum in a fermenter and then harvesting and purifying the fermented mixture in accordance with known procedures. All the botulinum toxin serotypes are initially synthesized as inactive single chain proteins which must be cleaved or nicked by proteases to become neuroactive. The bacterial strains that make botulinum toxin serotypes A and G possess endogenous proteases and serotypes A and G can therefore be recovered from bacterial cultures in predominantly their active form. In contrast, botulinum toxin serotypes C₁, D and E are synthesized by nonproteolytic strains and are therefore typically unactivated when recovered from culture. Serotypes B and F are produced by both proteolytic and nonproteolytic strains and therefore can be recovered in either the active or inactive form. However, even the proteolytic strains that produce, for example, the botulinum toxin type B serotype only cleave a portion of the toxin produced. The exact proportion of nicked to unnicked molecules depends on the length of incubation and the temperature of the culture. Therefore, a certain percentage of any preparation of, for example, the botulinum toxin type B toxin is likely to be inactive, possibly accounting for the known significantly lower potency of botulinum toxin type B as compared to botulinum toxin type A. The presence of inactive botulinum toxin molecules in a clinical preparation will contribute to the overall protein load of the preparation, which has been linked to increased antigenicity, without contributing to its clinical efficacy. Additionally, it is known that botulinum toxin type B has, upon intramuscular injection, a shorter duration of activity and is also less potent than botulinum toxin type A at the same dose level.

High quality crystalline botulinum toxin type A can be produced from the Hall A strain of Clostridium botulinum with characteristics of ≧3×10⁷ U/mg, an A₂₆₀/A₂₇₈ of less than 0.60 and a distinct pattern of banding on gel electrophoresis. The known Schantz process can be used to obtain crystalline botulinum toxin type A, as set forth in Schantz, E. J., et al, Properties and use of Botulinum toxin and Other Microbial Clostridial toxins in Medicine, Microbiol Rev. 56;80-99:1992. Generally, the botulinum toxin type A complex can be isolated and purified from an anaerobic fermentation by cultivating Clostridium botulinum type A in a suitable medium. The known process can also be used, upon separation out of the non-toxin proteins, to obtain pure botulinum toxins, such as for example: purified botulinum toxin type A with an approximately 150 kD molecular weight with a specific potency of 1-2×10⁸ LD₅₀ U/mg or greater; purified botulinum toxin type B with an approximately 156 kD molecular weight with a specific potency of 1-2×10⁸ LD₅₀ U/mg or greater, and; purified botulinum toxin type F with an approximately 155 kD molecular weight with a specific potency of 1-2×10⁷ LD₅₀ U/mg or greater.

Either the pure botulinum toxin (i.e. the 150 kilodalton botulinum toxin molecule) or the toxin complex can be used to prepare a pharmaceutical composition. Additionally retargeted botulinum toxin derived molecules with a high affinity for particular pain receptors can be used. See eg. Foster K., et al., Re-engineering the target specificity of clostridial neurotoxins: A route to novel therapeutics, Neurotox Res 2006 April;9(2-3):101-107. Both molecule and complex are susceptible to denaturation due to surface denaturation, heat, and alkaline conditions. Inactivated toxin forms toxoid proteins which may be immunogenic. The resulting antibodies can render a patient refractory to toxin injection.

As with enzymes generally, the biological activities of the botulinum toxins (which are intracellular peptidases) is dependant, at least in part, upon their three dimensional conformation. Thus, botulinum toxin type A is detoxified by heat, various chemicals surface stretching and surface drying. Additionally, it is known that dilution of the toxin complex obtained by the known culturing, fermentation and purification to the much, much lower toxin concentrations used for pharmaceutical composition formulation results in rapid detoxification of the toxin unless a suitable stabilizing agent is present. Dilution of the toxin from milligram quantities to a solution containing nanograms per milliliter presents significant difficulties because of the rapid loss of specific toxicity upon such great dilution. Since the toxin may be used months or years after the toxin containing pharmaceutical composition is formulated, the toxin can stabilized with a stabilizing agent such as albumin and gelatin.

A commercially available botulinum toxin containing pharmaceutical composition is sold under the trademark BOTOX® (available from Allergan, Inc., of Irvine, Calif.). BOTOX® consists of a purified botulinum toxin type A complex, albumin and sodium chloride packaged in sterile, vacuum-dried form. The botulinum toxin type A is made from a culture of the Hall strain of Clostridium botulinum grown in a medium containing N-Z amine and yeast extract. The botulinum toxin type A complex is purified from the culture solution by a series of acid precipitations to a crystalline complex consisting of the active high molecular weight toxin protein and an associated hemagglutinin protein. The crystalline complex is re-dissolved in a solution containing saline and albumin and sterile filtered (0.2 microns) prior to vacuum-drying. The vacuum-dried product is stored in a freezer at or below −5° C. BOTOX® can be reconstituted with sterile, non-preserved saline prior to intramuscular injection. Each vial of BOTOX® contains about 100 units (U) of Clostridium botulinum toxin type A purified Clostridial toxin complex, 0.5 milligrams of human serum albumin and 0.9 milligrams of sodium chloride in a sterile, vacuum-dried form without a preservative.

To reconstitute vacuum-dried BOTOX®, sterile normal saline without a preservative; (0.9% Sodium Chloride Injection) is used by drawing up the proper amount of diluent in the appropriate size syringe. Since BOTOX® may be denatured by bubbling or similar violent agitation, the diluent is gently injected into the vial. For sterility reasons BOTOX® is preferably administered within four hours after the vial is removed from the freezer and reconstituted. During these four hours, reconstituted BOTOX® can be stored in a refrigerator at about 2° C. to about 8° C. Reconstituted, refrigerated BOTOX® has been reported to retain its potency for at least about two weeks. Neurology, 48:249-53:1997.

Botulinum toxins have been used in clinical settings for the treatment of neuromuscular disorders characterized by hyperactive skeletal muscles. Botulinum toxin type A (BOTOX®) was approved by the U.S. Food and Drug Administration in 1989 for the treatment of essential blepharospasm, strabismus and hemifacial spasm in patients over the age of twelve. In 2000 the FDA approved commercial preparations of type A (BOTOX®) and type B botulinum toxin (MyoBloc™) serotypes for the treatment of cervical dystonia, and in 2002 the FDA approved a type A botulinum toxin (BOTOX®) for the cosmetic treatment of certain hyperkinetic (glabellar) facial wrinkles. Clinical effects of peripheral intramuscular botulinum toxin type A are usually seen within one week of injection and sometimes within a few hours. The typical duration of symptomatic relief (i.e. flaccid muscle paralysis) from a single intramuscular injection of botulinum toxin type A can be about three months, although in some cases the effects of a botulinum toxin induced denervation of a gland, such as a salivary gland, have been reported to last for several years. For example, it is known that botulinum toxin type A can have an efficacy for up to 12 months (Naumann M., et al., Botulinum toxin type A in the treatment of focal, axillary and palmar hyperhidrosis and other hyperhidrotic conditions, European J. Neurology 6 (Supp 4): S111-S115:1999), and in some circumstances for as long as 27 months. Ragona, R. M., et al., Management of parotid sialocele with botulinum toxin, The Laryngoscope 109:1344-1346:1999. However, the usual duration of an intramuscular injection of BOTOX® is typically about 3 to 4 months.

It has been reported that a botulinum toxin type A has been used in diverse clinical settings, including for example as follows:

(1) about 75-125 units of BOTOX® per intramuscular injection (multiple muscles) to treat cervical dystonia;

(2) 5-10 units of BOTOX® per intramuscular injection to treat glabellar lines (brow furrows) (5 units injected intramuscularly into the procerus muscle and 10 units injected intramuscularly into each corrugator supercilii muscle);

(3) about 30-80 units of BOTOX® to treat constipation by intrasphincter injection of the puborectalis muscle;

(4) about 1-5 units per muscle of intramuscularly injected BOTOX® to treat blepharospasm by injecting the lateral pre-tarsal orbicularis oculi muscle of the upper lid and the lateral pre-tarsal orbicularis oculi of the lower lid.

(5) to treat strabismus, extraocular muscles have been injected intramuscularly with between about 1-5 units of BOTOX®, the amount injected varying based upon both the size of the muscle to be injected and the extent of muscle paralysis desired (i.e. amount of diopter correction desired).

(6) to treat upper limb spasticity following stroke by intramuscular injections of BOTOX® into five different upper limb flexor muscles, as follows:
(a) flexor digitorum profundus: 7.5 U to 30 U
(b) flexor digitorum sublimus: 7.5 U to 30 U
(c) flexor carpi ulnaris: 10 U to 40 U
(d) flexor carpi radialis: 15 U to 60 U
(e) biceps brachii: 50 U to 200 U. Each of the five indicated muscles has been injected at the same treatment session, so that the patient receives from 90 U to 360 U of upper limb flexor muscle BOTOX® by intramuscular injection at each treatment session.

(7) to treat migraine, pericranial injected (injected symmetrically into glabellar, frontalis and temporalis muscles) injection of 25 U of BOTOX® has showed significant benefit as a prophylactic treatment of migraine compared to vehicle as measured by decreased measures of migraine frequency, maximal severity, associated vomiting and acute medication use over the three month period following the 25 U injection.

Additionally, intramuscular botulinum toxin has been used in the treatment of tremor in patients with Parkinson's disease, although it has been reported that results have not been impressive. Marjama-Lyons, J., et al., Tremor-Predominant Parkinson's Disease, Drugs & Aging 16(4);273-278:2000.

Treatment of certain gastrointestinal and smooth muscle disorders with a botulinum toxin are known. See e.g. U.S. Pat. Nos. 5,427,291 and 5,674,205 (Pasricha). Additionally, transurethral injection of a botulinum toxin into a bladder sphincter to treat a urination disorder is known (see e.g. Dykstra, D. D., et al, Treatment of detrusor-sphincter dyssynergia with botulinum A toxin: A double-blind study, Arch Phys Med Rehabil 1990 January;71:24-6), as is injection of a botulinum toxin into the prostate to treat prostatic hyperplasia. See e.g. U.S. Pat. No. 6,365,164 (Schmidt).

U.S. Pat. No. 5,766,605 (Sanders) proposes the treatment of various autonomic disorders, such as excessive stomach acid secretion, hypersalivation, rhinittis, with a botulinum toxin. Additionally, It is known that nasal hypersecretion is predominantly caused by over activity of nasal glands, which are mainly under cholinergic control and it has been demonstrated that application of botulinum toxin type A to mammalian nasal mucosal tissue of the maxillary sinus turbinates can induce a temporary apoptosis in the nasal glands. Rohrbach S., et al., Botulinum toxin type A induces apoptosis in nasal glands of guinea pigs, Ann Otol Rhinol Laryngol 2001 November;110(11):1045-50. Furthermore, local application of botulinum toxin A to a human female patient with intrinsic rhinitis resulted in a clear decrease of the nasal hypersecretion within five days. Rohrbach S., et al., Minimally invasive application of botulinum toxin type A in nasal hypersecretion, J Oto-Rhino-Laryngol 2001 November-December;63(6):382-4.

Various afflictions, such as hyperhydrosis and headache, treatable with a botulinum toxin are discussed in WO 95/17904 (PCT/US94/14717) (Aoki). EP 0 605 501 B1 (Graham) discusses treatment of cerebral palsy with a botulinum toxin and U.S. Pat. No. 6,063,768 (First) discusses treatment of neurogenic inflammation with a botulinum toxin.

In addition to having pharmacologic actions at the peripheral location, botulinum toxins can also have inhibitory effects in the central nervous system. Work by Weigand et al, (¹²⁵I-labelled botulinum A Clostridial toxin:pharmacokinetics in cats after intramuscular injection, Naunyn-Schmiedeberg's Arch. Pharmacol. 1976; 292, 161-165), and Habermann, (¹²⁵I-labelled Clostridial toxin from clostridium botulinum A: preparation, binding to synaptosomes and ascent to the spinal cord, Naunyn-Schmiedeberg's Arch. Pharmacol. 1974; 281, 47-56) showed that botulinum toxin is able to ascend to the spinal area by retrograde transport. As such, a botulinum toxin injected at a peripheral location, for example intramuscularly, may be retrograde transported to the spinal cord.

In vitro studies have indicated that botulinum toxin inhibits potassium cation induced release of both acetylcholine and norepinephrine from primary cell cultures of brainstem tissue. Additionally, it has been reported that botulinum toxin inhibits the evoked release of both glycine and glutamate in primary cultures of spinal cord neurons and that in brain synaptosome preparations botulinum toxin inhibits the release of each of the neurotransmitters acetylcholine, dopamine, norepinephrine, CGRP and glutamate.

U.S. Pat. No. 5,989,545 discloses that a modified Clostridial toxin or fragment thereof, preferably a botulinum toxin, chemically conjugated or recombinantly fused to a particular targeting moiety can be used to treat pain by administration of the agent to the spinal cord.

A botulinum toxin has also been proposed for the treatment of hyperhydrosis (excessive sweating, U.S. Pat. No. 5,766,605), headache, (U.S. Pat. No. 6,458,365, migraine headache (U.S. Pat. No. 5,714,468), post-operative pain and visceral pain (U.S. Pat. No. 6,464,986), pain by intraspinal administration (U.S. Pat. No. 6,113,915), Parkinson's disease by intracranial administration (U.S. Pat. No. 6,306,403), hair growth and hair retention (U.S. Pat. No. 6,299,893), psoriasis and dermatitis (U.S. Pat. No. 5,670,484), injured muscles (U.S. Pat. No. 6,423,319, various cancers (U.S. Pat. No. 6,139,845), pancreatic disorders (U.S. Pat. No. 6,143,306), smooth muscle disorders (U.S. Pat. No. 5,437,291, including injection of a botulinum toxin into the upper and lower esophageal, pyloric and anal sphincters), prostate disorders (U.S. Pat. No. 6,365,164), inflammation, arthritis and gout (U.S. Pat. No. 6,063,768), juvenile cerebral palsy (U.S. Pat. No. 6,395,277), inner ear disorders (U.S. Pat. No. 6,265,379), thyroid disorders (U.S. Pat. No. 6,358,513), parathyroid disorders (U.S. Pat. No. 6,328,977). Additionally, controlled release toxin implants are known (U.S. Pat. Nos. 6,306,423 and 6,312,708).

Botulinum toxin has been proposed for the treatment of headache pain (U.S. Pat. No. 6,458,365), migraine headache pain (U.S. Pat. No. 5,714,468), post-operative pain and visceral pain (U.S. Pat. No. 6,464,986). The pain associated with a tattoo placement is unique. For example, the pain associated with a tattoo placement is described as being a “tingling pain” or “stinging pain”. The pain associated with a tattoo removal (when using a laser, for example) is described as similar to that of being “snapped” with a rubber band, or a “snapping” pain.

None of the prior references teaches the administration of a botulinum toxin to treat/alleviate a pain associated with a placement or removal of a tattoo. More specifically, none of the prior references teaches the administration a botulinum toxin to the skin (e.g., intradermally) to alleviate a pain associated with the placement or removal of a tattoo.

Tetanus toxin, as wells as derivatives (i.e. with a non-native targeting moiety), fragments, hybrids and chimeras thereof can also have therapeutic utility. The tetanus toxin bears many similarities to the botulinum toxins. Thus, both the tetanus toxin and the botulinum toxins are polypeptides made by closely related species of Clostridium (Clostridium tetani and Clostridium botulinum, respectively). Additionally, both the tetanus toxin and the botulinum toxins are dichain proteins composed of a light chain (molecular weight about 50 kD) covalently bound by a single disulfide bond to a heavy chain (molecular weight about 100 kD). Hence, the molecular weight of tetanus toxin and of each of the seven botulinum toxins (non-complexed) is about 150 kD. Furthermore, for both the tetanus toxin and the botulinum toxins, the light chain bears the domain which exhibits intracellular biological (protease) activity, while the heavy chain comprises the receptor binding (immunogenic) and cell membrane translocational domains.

Further, both the tetanus toxin and the botulinum toxins exhibit a high, specific affinity for gangliocide receptors on the surface of presynaptic cholinergic neurons. Receptor mediated endocytosis of tetanus toxin by peripheral cholinergic neurons results in retrograde axonal transport, blocking of the release of inhibitory neurotransmitters from central synapses and a spastic paralysis. Contrarily, receptor mediated endocytosis of botulinum toxin by peripheral cholinergic neurons results in little if any retrograde transport, inhibition of acetylcholine exocytosis from the intoxicated peripheral motor neurons and a flaccid paralysis.

Finally, the tetanus toxin and the botulinum toxins resemble each other in both biosynthesis and molecular architecture. Thus, there is an overall 34% identity between the protein sequences of tetanus toxin and botulinum toxin type A, and a sequence identity as high as 62% for some functional domains. Binz T. et al., The Complete Sequence of Botulinum Clostridial toxin Type A and Comparison with Other Clostridial toxins, J Biological Chemistry 265(16);9153-9158:1990.

Acetylcholine

Typically only a single type of small molecule neurotransmitter is released by each type of neuron in the mammalian nervous system. The neurotransmitter acetylcholine is secreted by neurons in many areas of the brain, but specifically by the large pyramidal cells of the motor cortex, by several different neurons in the basal ganglia, by the motor neurons that innervate the skeletal muscles, by the preganglionic neurons of the autonomic nervous system (both sympathetic and parasympathetic), by the postganglionic neurons of the parasympathetic nervous system, and by some of the postganglionic neurons of the sympathetic nervous system. Essentially, only the postganglionic sympathetic nerve fibers to the sweat glands, the piloerector muscles and a few blood vessels are cholinergic as most of the postganglionic neurons of the sympathetic nervous system secret the neurotransmitter norepinephrine. In most instances acetylcholine has an excitatory effect. However, acetylcholine is known to have inhibitory effects at some of the peripheral parasympathetic nerve endings, such as inhibition of heart rate by the vagal nerve.

The efferent signals of the autonomic nervous system are transmitted to the body through either the sympathetic nervous system or the parasympathetic nervous system. The preganglionic neurons of the sympathetic nervous system extend from preganglionic sympathetic neuron cell bodies located in the intermediolateral horn of the spinal cord. The preganglionic sympathetic nerve fibers, extending from the cell body, synapse with postganglionic neurons located in either a paravertebral sympathetic ganglion or in a prevertebral ganglion. Since the preganglionic neurons of both the sympathetic and parasympathetic nervous system are cholinergic, application of acetylcholine to the ganglia will excite both sympathetic and parasympathetic postganglionic neurons.

Acetylcholine activates two types of receptors, muscarinic and nicotinic receptors. The muscarinic receptors are found in all effector cells stimulated by the postganglionic, neurons of the parasympathetic nervous system as well as in those stimulated by the postganglionic cholinergic neurons of the sympathetic nervous system. The nicotinic receptors are found in the adrenal medulla, as well as within the autonomic ganglia, that is on the cell surface of the postganglionic neuron at the synapse between the preganglionic and postganglionic neurons of both the sympathetic and parasympathetic systems. Nicotinic receptors are also found in many nonautonomic nerve endings, for example in the membranes of skeletal muscle fibers at the neuromuscular junction.

Acetylcholine is released from cholinergic neurons when small, clear, intracellular vesicles fuse with the presynaptic neuronal cell membrane. A wide variety of non-neuronal secretory cells, such as, adrenal medulla (as well as the PC12 cell line) and pancreatic islet cells release catecholamines and parathyroid hormone, respectively, from large dense-core vesicles. The PC12 cell line is a clone of rat pheochromocytoma cells extensively used as a tissue culture model for studies of sympathoadrenal development. Botulinum toxin inhibits the release of both types of compounds from both types of cells in vitro, permeabilized (as by electroporation) or by direct injection of the toxin into the denervated cell. Botulinum toxin is also known to block release of the neurotransmitter glutamate from cortical synaptosomes cell cultures.

A neuromuscular junction is formed in skeletal muscle by the proximity of axons to muscle cells. A signal transmitted through the nervous system results in an action potential at the terminal axon, with activation of ion channels and resulting release of the neurotransmitter acetylcholine from intraneuronal synaptic vesicles, for example at the motor endplate of the neuromuscular junction. The acetylcholine crosses the extracellular space to bind with acetylcholine receptor proteins on the surface of the muscle end plate. Once sufficient binding has occurred, an action potential of the muscle cell causes specific membrane ion channel changes, resulting in muscle cell contraction. The acetylcholine is then released from the muscle cells and metabolized by cholinesterases in the extracellular space. The metabolites are recycled back into the terminal axon for reprocessing into further acetylcholine.

As discussed above, the conventional procedures for treating pain associated with the placement or removal of a tattoo are inadequate because, for example, the pain relief is not long lasting.

What is needed therefore is an improved method for alleviating pain associated with a tattoo placement or tattoo removal.

### SUMMARY

The present invention meets this need and provides for improved methods for alleviating a pain associated with a placement or removal of a tattoo. In one embodiment a method for alleviating a pain associated with placement or removal of a tattoo comprises the step of locally administering a botulinum toxin to alleviate the pain associated with placement or removal of a tattoo. The pain can be a tingling pain or a snapping pain. The botulinum toxin can be administered to the skin. Thus, the botulinum toxin can be administered transdermally, subcutaneously or intradermally. The tattoo can be removed using a laser, dermabrasion, salabrasion or an excision procedure.

My invention includes a method wherein the botulinum toxin is administered about 1 week to about 4 weeks prior to a tattoo placement or removal. The botulinum toxin used in my invention is selected from the group consisting of botulinum toxins types A, B, C₁, D, E, F and G. Preferably the botulinum toxin is a botulinum toxin type A, such as a botulinum toxin to type A₁ or A₂ or a suitable retargeted botulinum toxin derived molecule.

A preferred method within the scope of my invention is a method for alleviating a tingling pain associated with placement of a tattoo by locally administering a botulinum toxin to alleviate the tingling pain associated with placement of a tattoo.

Another method within the scope of my invention is a method for alleviating a snapping pain associated with a laser removal of a tattoo by locally administering a botulinum toxin to alleviate the snapping pain associated with the laser removal of a tattoo.

In some embodiments, the dose of neurotoxin to be administered is equivalent to about 1 unit to about 500 units of a botulinum toxin type A.

The term “neurotoxin” employed herein refers to one or more of a toxin made by a bacterium, for example, a Clostridium botulinum, Clostridium butyricum, Clostridium beratti, Clostridium tetani. In some embodiments, the neurotoxin is a botulinum toxin. The botulinum toxin may be a botulinum toxin type A (including A₁ or A₂), type B, type C₁, type D, type E, type F, or type G. In some embodiments, the neurotoxin is a botulinum toxin type A. Unless stated otherwise, the dose of the neurotoxin referenced herein is equivalent to that of a botulinum toxin type A. The assays required to determine equivalency to the therapeutic effectiveness of botulinum toxin type A at a certain dosage are well established.

Further, the botulinum toxin of the present invention may comprise a first element comprising a binding element able to specifically bind to a neuronal cell surface receptor under physiological conditions; a second element comprising a translocation element able to facilitate the transfer of a polypeptide across a neuronal cell membrane, and a third element comprising a therapeutic element able, when present in the cytoplasm of a neuron, to inhibit exocytosis of acetylcholine from the neuron. The therapeutic element can cleave a SNARE protein, thereby inhibiting the exocytosis of acetylcholine from the neuron. The SNARE protein can be selected from the group consisting of syntaxin, SNAP-25 and VAMP.

### DEFINITIONS

The following definitions apply herein.

“About” means plus or minus ten percent of the value so qualified.

“Alleviate” as applied to pain means a reduction of pain. In some embodiments, the reduction of pain is reduced by more than 25%. In some embodiments, the reduction of pain is by more than 50%. The reduction of pain is measured by the patient reporting the degree of pain after the neurotoxin treatment as compared to the degree of pain prior to the treatment.

“Effective amount” as applied to the neurotoxin means that amount of the neurotoxin generally sufficient to effect a desired change in the subject. For example, where the desired effect is decreasing the pain of a tattoo placement or removal, an effective amount of the compound is that amount which causes at least a decrease in the spasm of the vas deferens by more than 30%. In some embodiments, the neurotoxin can be administered in an amount between about 0.01 U/kg and about 35 U/kg and the pain treated can be substantially alleviated for between about 1 month and about 27 months, for example for from about 1 month to about 6 months.

“Locally administering” or “local administration” means direct injection to a tissue, e.g., subcutaneous, intradermal administration. Local administration excludes systemic routes of administration, such as intravenous or oral administration.

“Transdermal” means applied directly to the surface of the skin, e.g., by means of a patch or ointment containing suitable ingredients (e.g., permeation enhancers) and/or engineering (e.g., microneedles) to facilitate penetration through the dermis barrier and may be combined with other synergistic therapies (e.g., iontophoresis).

### DESCRIPTION

The present invention is partly based upon the surprising discovery that an administration of a neurotoxin, such as a botulinum toxin type A or other types, to a skin area can alleviate a pain that the patient experiences from a tattoo placement or removal.

In some embodiments, the methods comprise the step of locally administering a neurotoxin (e.g., a botulinum toxin).

Treatments such as salabrasion and dermabrasion surgery can lighten and sometimes fully clear tattoos, but these and other destructive modalities can cause permanent scarring. Laser tattoo removal is the treatment of choice for removal of amateur and professional tattoos of all colors and in all skin types. Because of the small size of tattoo particles, pulses in the nanosecond domain are required for tattoo removal. Q-SWITCHED lasers deliver pulses in the nanosecond domain and are the optimal devices for tattoo removal.

In some embodiments, the dose of a neurotoxin administered is equivalent to about 1 unit to about 500 units of a botulinum toxin type A. In some embodiments, the dose of a neurotoxin administered is equivalent to about 1 unit to about 300 units of a botulinum toxin type A. In some embodiments, the dose of a neurotoxin administered is equivalent to about 1 unit to about 150 units of a botulinum toxin type A. In some embodiments, the dose of a neurotoxin administered is equivalent to about 1 unit to about 75 units of a botulinum toxin type A. In some embodiments, the dose of a neurotoxin administered is equivalent to about 1 unit to about 40 units of a botulinum toxin type A. In some embodiments, the dose of a neurotoxin administered is in an amount of between about 0.1 unit and about 5 units.

In some embodiments, a neurotoxin, such as a botulinum toxin type A or other types, can be locally administered according to the present disclosed methods in amounts of between about 10⁻³ U/kg to about 10 U/kg. In some embodiments, a neurotoxin, such as a botulinum toxin type A or other types, can be locally administered according to the present disclosed methods in amounts of between about 10⁻² U/kg and about 1 U/kg. In some embodiments, a neurotoxin, such as a botulinum toxin type A or other types, can be locally administered according to the present disclosed methods in amounts of between about 10⁻¹ U/kg and about 1 U/kg. Significantly, the pain alleviating effect of the present disclosed methods can persist for between about 2 months to about 6 months.

Methods for determining the appropriate route of administration and dosage are generally determined on a case by case basis by the attending physician. Such determinations are routine to one of ordinary skill in the art (see for example, Harrison's Principles of Internal Medicine (1998), edited by Anthony Fauci et al., 14^{th} edition, published by McGraw Hill).

In some embodiments, the neurotoxin is administered to a “patient in need thereof”, which means that the patient has been specifically diagnosed as having a testicle pain and the neurotoxin is administered for the specific purpose of alleviating the testicular pain.

Preferably, a neurotoxin used to practice a method within the scope of the present invention is a botulinum toxin, such as one of the serotype A (including A₁ and A₂), B, C, D, E, F or G botulinum toxins. Preferably, the botulinum toxin used is botulinum toxin type A, because of its high potency in humans, ready availability, and known safe and efficacious use for the treatment of skeletal muscle and smooth muscle disorders when locally administered by intramuscular injection.

The present invention includes within its scope: (a) Clostridial toxin complex as well as pure Clostridial toxin obtained or processed by bacterial culturing, toxin extraction, concentration, preservation, freeze drying and/or reconstitution and; (b) modified or recombinant Clostridial toxin, that is Clostridial toxin that has had one or more amino acids or amino acid sequences deliberately deleted, modified or redeployed by known chemical/biochemical amino acid modification procedures or by use of known host cell/recombinant vector recombinant technologies, as well as derivatives or fragments of Clostridial toxins so made, and includes Clostridial toxins with one or more attached targeting moieties for a cell surface receptor present on a cell.

Neurotoxins, e.g., botulinum toxins, for use according to the present invention can be stored in liquids, creams, ointments, lyophilized or vacuum dried form in containers. Prior to lyophilization the botulinum toxin can be combined with pharmaceutically acceptable excipients, stabilizers and/or carriers, such as albumin. Ointments or creams can be applied to a substrate such as a patch or used independently. The lyophilized or vacuum dried material can be reconstituted with saline or water.

### EXAMPLES

The following examples set forth specific methods encompassed by the present invention and are not intended to limit the scope of the present invention.

### Example 1

### Methods for Alleviating Pain Associated with a Placement of a Tattoo

A 21 year old man is interested in procuring a tattoo on his ankle. He contacts his physician to have botulinum toxin administered to the area to be tattooed. The botulinum toxin is administered to the ankle about one week prior to the tattoo being placed there.

Within the area to be tattooed, the botulinum toxin is injected (e.g., intradermally) at about 1 cm apart. During the tattoo session the man experiences minimal pain.

The healing process for a tattoo procedure takes about four weeks. During the healing process it is normal to have some pain, discomfort, and itchiness. The botulinum toxin also blocks the pain, discomfort, and itchiness during the healing process.

### Example 2

### Methods for Alleviating Pain Associated with a Laser Procedure to Remove a Tattoo

A 30-year-old white woman with Fitzpatrick skin type I presents for removal of a black tattoo on her back. The patient is treated with the SINON Q-SWITCHED RUBY laser. About a week prior to the laser sessions, the treatment site is administered with a therapeutically effective amount of botulinum toxin. Preferably, the botulinum toxin is administered intradermally at the location to be treated with the laser. In some embodiments, the injection locations of the botulinum toxin are spaced at about 1 cm apart.

A clear hydrogel dressing is applied (with the plastic barrier removed from both sides) to protect the epidermis, reduce bleeding and skin fragmentation, and prevent aerosolization of skin fragments and blood. The patient undergoes 7 laser treatments administered at 6-week intervals. The fluence is gradually increased over the course of treatment from a starting level of 2 J/cm² to a maximum of 6 J/cm² (average fluence, 5.1 J/cm²). A 5-mm spot size is used, and treatments are administered with a repetition rate of 2 pulses per second. Following laser treatment, the hydrogel dressing is taped in place, and the patient is instructed to apply a healing ointment along with a nonstick bandage held in place with paper tape.

Treatment of the botulinum toxin results in the patient not feeling any pain during the laser treatment procedures and throughout the healing period of the treated location.

### Example 3

### Methods for Alleviating Pain Associated with a Laser Procedure to Remove a Nevus of Ota

Nevus of Ota is a relatively uncommon dark discolorations occurring around the eye and shoulders. An 18-month-old girl with extensive nevus of Ota underwent 3 treatments with the SINON Q-SWITCHED RUBY laser over the course of 9 months. About 5 days prior to the laser treatment sessions, the treatment site is administered with a therapeutically effective amount of botulinum toxin. Preferably, the botulinum toxin is administered intradermally at the location to be treated with the laser.

Fluence ranges from 4.5 to 6.0 J/cm², and a 5-mm spot size was used. Sedation was not needed, as the child was held during each session.

Treatment of the botulinum toxin results in the child not feeling any pain during the laser treatment procedures and throughout the healing period of the treated location.

All references, articles, publications and patents and patent applications cited herein are incorporated by reference in their entireties.

Although the present invention has been described in detail with regard to certain preferred methods, other embodiments, versions, and modifications within the scope of the present invention are possible. For example, a wide variety of Clostridial toxins can be effectively used in the methods of the present invention. Additionally, the present invention includes formulations where two or more botulinum toxins, are administered concurrently or consecutively. For example, botulinum toxin type A can be administered until a loss of clinical response or neutralizing antibodies develop, followed by administration also by a botulinum toxin type B or E. Alternately, a combination of any two or more of the botulinum serotypes A-G can be locally administered to control the onset and duration of the desired therapeutic result. Furthermore, non-Clostridial toxin compounds can be administered prior to, concurrently with or subsequent to administration of the Clostridial toxin so as to provide an adjunct effect such as enhanced or a more rapid onset of denervation before the Clostridial toxin, such as a botulinum toxin, begins to exert its therapeutic effect.

Accordingly, the spirit and scope of the following claims should not be limited to the descriptions of the preferred embodiments set forth above.

What is claimed is:

1. A method for alleviating a pain associated with placement or removal of a tattoo, the method comprising the step of locally administering a botulinum toxin to alleviate the pain associated with placement or removal of a tattoo.

2. The method of claim 1 wherein the pain is a tingling pain.

3. The method of claim 1 wherein the pain is a snapping pain.

4. The method of claim 1 wherein the botulinum toxin is administered to the skin.

5. The method of claim 1 wherein the botulinum toxin is administered subcutaneously.

6. The method of claim 1 wherein the botulinum toxin is administered intradermally.

7. The method of claim 1 wherein the botulinum toxin is administered about 1 week to about 4 weeks prior to a tattoo placement or removal.

8. The method of claim 1 wherein the botulinum toxin is selected from the group consisting of botulinum toxins types A, B, C₁, D, E, F and G.

9. The method of claim 1 wherein the botulinum toxin is a botulinum toxin type A.

10. The method of claim 1 wherein the botulinum toxin is a botulinum toxin to type A₁ or A₂.

11. The method of claim 1 wherein the tattoo removal is via the use of a laser, dermabrasion, salabrasion or excision procedure.

12. The method of claim 1 wherein the tattoo removal is via the use of a laser procedure.

13. A method for alleviating a tingling pain associated with placement of a tattoo, the method comprising the step of locally administering a botulinum toxin to alleviate the tingling pain associated with placement of a tattoo.

14. The method of claim 13 wherein the botulinum toxin is administered to the skin.

15. The method of claim 13 wherein the botulinum toxin is administered subcutaneously.

16. The method of claim 13 wherein the botulinum toxin is administered intradermally.

17. A method for alleviating a snapping pain associated with a laser removal of a tattoo, the method comprising the step of locally administering a botulinum toxin to alleviate the snapping pain associated with the laser removal of a tattoo.

18. The method of claim 17 wherein the botulinum toxin is administered to the skin.

19. The method of claim 17 wherein the botulinum toxin is administered subcutaneously.

20. The method of claim 17 wherein the botulinum toxin is administered intradermally.

21. The method of claim 13 wherein the botulinum toxin is administered transdermally.

22. The method of claim 17 wherein the botulinum toxin is administered transdermally.
